(19) United States
(12) Patent Application Publication
(10) Pub. No.: US 2005/0065206 A1
(43) Pub. Date: Mar. 24, 2005

(54) Skin preparation comprising a tocopherol derivative for external application

(76) Inventors: Eiko Kato, Chiba-shi (JP); Toshi Tsuzuki, Chiba-shi (JP); Jiro Takata, Fukuoka-shi (JP); Yoshiharu Karube, Fukuoka-shi (JP); Kazuhisa Matsunaga, Fukuoka-shi (JP); Shizuko Kobayashi, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/493,695

(22) PCT Filed: Oct. 28, 2002
(86) PCT No.: PCT/JP02/11151

Related U.S. Application Data
(60) Provisional application No. 60/359,334, filed on Feb. 26, 2002.
(60) Provisional application No. 60/373,579, filed on Apr. 19, 2002.

(30) Foreign Application Priority Data
Oct. 29, 2001 (JP) 2001-331581
Apr. 12, 2002 (JP) 2002-110106

(51) Int. Cl. A61K 31/355; A61K 7/00

(52) U.S. Cl. 514/458; 424/401

(57) ABSTRACT

The present invention relates to a skin preparation for external application, comprising a tocopherol aminoalkylcarboxylate ester having no substituent on the N atom and/or a salt thereof. Preferable tocopherol aminoalkylcarboxylate in the present invention is one or more compound selected from α-tocopherol derivatives, β-tocopherol derivatives, γ-tocopherol derivatives and δ-tocopherol derivatives. The present invention also relates to a cosmetic material comprising the skin preparation.

This application is an application filed under 35 U.S.C. 111(a) claiming pursuant to 35 U.S.C. 119(e) of the filing date of Provisional Application 60/359,334 on Feb. 26, 2002, Provisional Application 60/373,579 on Apr. 19, 2002, pursuant to 35 U.S.C. 111 (b).

### TECHNICAL FIELD

The present invention relates to a skin preparation for external application and a cosmetic material, characterized in that a tocopherol aminoalkylcarboxylate ester having no substituent on the N atom and/or a salt thereof is blended.

### BACKGROUND ART

Tocopherols (e.g., α-tocopherol, β-tocopherol, γ-tocopherol, δ-todopherol) known as vitamin E and derivatives thereof such as tocopherol acetate and tocopherol nicotinate are known to provide efficacy and effect such as activities of antioxidation, vital membrane stabilization, immunoactivation and acceleration of blood circulation and have been long blended in medical preparations, cosmetics, feed and the like.

However, these compounds are oil-soluble and cannot be uniformly dispersed in an emulsion using an aqueous solution or water as the base. In the case of preparing a medical or cosmetic product in the solubilized or emulsion state, a nonionic surfactant is generally used to enable uniform dispersion, however, some nonionic surfactants are highly irritating or give rise to environmental pollution and therefore, in view of safety, use of the nonionic surfactant is considered undesirable and improvement is demanded in this point.

Furthermore, tocopherols in the simple form are readily oxidized and unstable and therefore, are used as an organic acid ester derivative such as acetate ester, nicotinate ester or succinate ester in many cases. In order to allow the organic acid ester derivative to exert in vivo the physiological activity as tocopherol, the ester bond moiety must be hydrolyzed by an enzyme such as esterase, however, the conversion rate of those derivatives is not sufficiently high and the effect of increasing the concentration in the tissue is low. Accordingly, development of a derivative which is more easily converted is being demanded.

As for the derivative of tocopherols, JP-A-58-203982 describes vitamin E-amino acid esters. However, the vitamin E-amino acid esters described are in an oil or wax state and it is easily inferred that the preparation of a medical or cosmetic product in the solubilized or emulsion state is difficult.

Furthermore, examples of the amino acid in the vitamin E-amino acid ester described in JP-A-58-203982 are only L-methionine, β-alanine, L-cysteine, L-cystine, valine, leucine, phenylalanine, serine, tyrosine, lysine, arginine, histidine and glutamic acid, but glycine is not referred to.

It is an object of the present invention to improve the solubility, emulsifiability and dispersibility of tocopherol in skin preparations for external application and provide a tocopherol aminoalkylcarboxylate ester having no substituent on the N atom and/or a salt thereof which undergo efficient conversion to active tocopherol in skin tissue.

### DISCLOSURE OF THE INVENTION

As a result of extensive investigations to overcome the above-described problems, the present inventors have found that a tocopherol aminoalkylcarboxylate ester having no substituent on the N atom and/or a salt thereof have useful solubility, emulsifiability and dispersibility, and have accomplished the present invention. As used herein, “having a substituent on the N atom” means to have a substituent other than an alkylcarboxylate on an amino group of the aminoalkylcarboxylate.

The present inventors have also found that the tocopherol aminoalkylcarboxylate ester having no substituent on the N atom and/or a salt thereof are efficiently converted to active tocopherol in skin tissue, and have accomplished the present invention.

More specifically, the present invention relates to the following matters.

[1] A skin preparation for external application, comprising a tocopherol aminoalkylcarboxylate ester having no substituent on the N atom and/or a salt thereof.

[2] The skin preparation for external application as described in [1] above, wherein the tocopherol aminoalkylcarboxylate ester is one or more compound selected from α-tocopherol derivatives, β-tocopherol derivatives, γ-tocopherol derivatives and δ-tocopherol derivatives.

[3] The skin preparation for external application as described in [2] above, wherein the tocopherol aminoalkylcarboxylate ester is an α-tocopherol aminoalkylcarboxylate ester or a γ-tocopherol aminoalkylcarboxylate ester.

[4] The skin preparation for external application as described in any one of [1] to [3] above, wherein the tocopherol aminoalkylcarboxylate ester having no substituent on the N atom comprises a compound represented by the following formula (I): 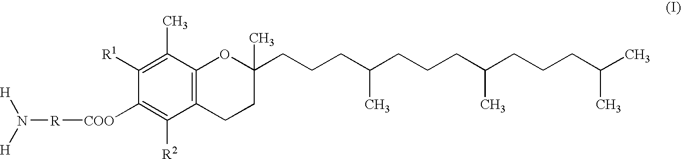
(wherein R¹ and R² each represents a hydrogen atom or a methyl group and R represents a branched or linear alkylene group which may have a substituent).

[5] The skin preparation for external application as described in any one of [1] to [4] above, wherein the tocopherol aminoalkylcarboxylate ester having no substituent on the N atom comprises a compound represented by the following formula (II): 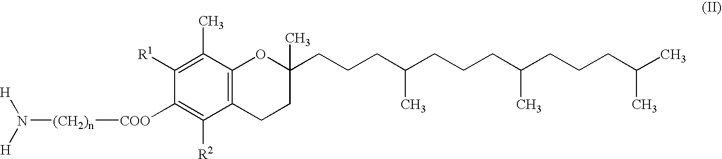
(wherein R¹ and R² each represents a hydrogen atom or a methyl group, and n represents an integer of 1 to 7).

[6] The skin preparation for external application as described in any one of [1] to [5] above, wherein the salt is a hydrohalogenic acid.

[7] The skin preparation for external application as described in any one of [1] to [5] above, wherein the salt is an organic acid salt.

[8] The skin preparation for external application as described in [6] above, wherein the hydrohalogenic acid is hydrochloric acid.

[9] The skin preparation for external application as described in any one of [1] to [8] above, wherein the content of the tocopherol aminoalkylcarboxylate ester having no substituent on the N atom and/or a salt thereof is from 0.01 to 10 mass %.

[10] The skin preparation for external application as described in any one of [1] to [9] above, wherein the tocopherol aminoalkylcarboxylate ester and/or a salt thereof is tocopherol glycine ester and/or a salt thereof.

[11] A cosmetic material comprising the skin preparation for external application described in any one of [1] to [10] above.

### BEST MODE FOR CARRYING OUT THE INVENTION

The tocopherol aminoalkylcarboxylate ester derivative having no substituent on the N atom of the present invention and the tocopherol aminoalkylcarboxylate ester having no substituent on the N atom and/or a salt thereof for use in the skin preparation for external application of the present invention are described below.

The tocopherol aminoalkylcarboxylate ester derivative having no substituent on the N atom is, for example, a compound represented by the following formula (I) and/or a salt thereof: 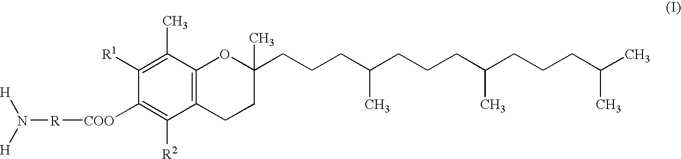
(wherein R¹ and R² each represents a hydrogen atom or a methyl group and R represents a branched or linear alkylene group which may have a substituent).

As seen from the formula above, the tocopherol which can be used in the present invention includes α-, β-, γ- and δ-tocopherol derivatives.

Among these, preferred are α-tocopherol where R¹ and R² are methyl, and γ-tocopherol where R¹ is methyl and R² is a hydrogen atom.

These tocopherol derivatives have an asymmetric carbon atom at the 2-position of the chromanol ring and therefore, steric isomers such as d form and dl form are present. Needless to say, the present invention includes all of these isomers.

In the tocopherol aminoalkylcarboxylate ester derivative having no substituent on the N atom of the present invention, the aminoalkylcarboxylic acid may be any of D form, L form and DL form but in view of bioactivity and the like, L form or DL form is preferred.

In the present invention, a salt is preferred and the salt is preferably a hydrohalogenic acid salt, more preferably an HCl salt or an HBr salt.

In particular, the HCl salt is advantageous in that the solubility in water increases and due to its powder form, handling is facilitated.

Examples of the organic acids which are also preferred as the salt for use in the present invention include an alkane acid, a linear alkene acid, a saturated dibasic acid, an alkane dipentaene acid, an alkane tripentaene acid, an alkane tetrapentaene acid, a hydroxystearic acid, an α-hydroxy acid and an amino acid.

Among the tocopherol aminoalkylcarboxylate esters having no substituent on the N atom and/or salts thereof contained in the skin preparation for external application of the present invention, a tocopherol glycine ester and/or a salt thereof are suitably used.

The tocopherol aminoalkylcarboxylate ester derivative having no substituent on the N atom of the present invention may be produced by various methods, but a representative method is described below.

The representative production method is described by referring to the case of R═(CH₂)ₙ (wherein n represents an integer of 1 to 7) which is a preferred example.

This compound can be easily obtained by performing an esterification reaction of a tocopherol represented by formula (III): 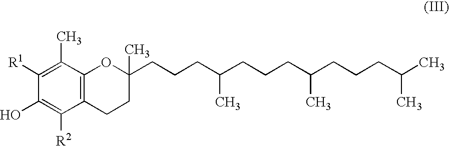
(wherein R¹ and R² each represents a hydrogen atom or a methyl group) and any one of an aminoalkylcarboxylic acid represented by the following formula (IV): 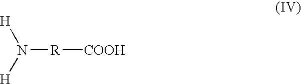
(wherein R represents a branched or linear alkylene group which may have a substituent), its reactive acid derivative and a salt thereof such as hydrohalogenic acid salt or organic acid salt, in a usual manner.

In the case of directly performing the esterification using a free aminoalkylcarboxylic acid, usually, the reaction is preferably performed in the presence of an active esterification reagent (dehydrating agent) such as dicyclohexylcarbodiimide and N,N-disuccinimide oxalate. At this time, the solvent is most preferably pyridine.

If desired, the aminoalkylcarboxylic acid having no substituent on the N atom after the completion of reaction is preferably subjected to a treatment for removing the protective group using an aminoalkylcarboxylic acid in which the amino group is protected, for example, by an N-tert-butoxycarbonyl (BOC) group, a benzyloxycarbonyl group or a 2-nitrobenzenesulfonyl group.

In the method of using a reactive acid derivative, an acid halide, particularly acid chloride is preferably used.

In the case of producing a hydrohalogenic acid salt of a tocopherol aminoalkylcarboxylate ester having no substituent on the N atom, the hydrohalogenic acid salt may be produced by once producing an ester form and reacting it with a hydrohalogenic acid (gas phase or solution) in a usual manner, or a hydrohalogenic acid salt of an aminoalkylcarboxylic acid having no substituent on the N atom as represented by formula (IV) may be previously used as a starting material.

In the case of producing an organic acid salt of a tocopherol aminoalkylcarboxylate ester having no substituent on the N atom, an organic acid salt may be produced by once producing an ester form and reacting it with an organic acid (gas phase or solution) in a usual manner, or an organic acid salt of an aminoalkylcarboxylic acid having no substituent on the N atom may be previously used as a starting material.

The thus-obtained tocopherol aminoalkylcarboxylate ester having no substituent on the N atom and/or a salt thereof are excellent in the solubility and emulsifiability as compared with tocopherols in a simple form. Furthermore, when applied as a skin preparation for external application, these are readily hydrolyzed by an esterase or carboxyl esterase in the skin tissue to produce an active free tocopherol.

Therefore, the tocopherol aminoalkylcarboxylate ester having no substituent on the N atom and/or a salt thereof of the present invention can be used as an active ingredient of skin preparations for external application which are expected to have efficacy and effect such as activities of antioxidation, vital membrane stabilization, immunoactivation and acceleration of blood circulation.

The skin preparation for external application of the present invention is a skin preparation for external application where a tocopherol aminoalkylcarboxylate ester having no substituent on the N atom and/or a salt thereof is blended and particularly when a hydrohalogenic acid salt is blended, the skin preparation for external application can be suitably used as a cosmetic material.

The cosmetic material of the present invention includes, in a wide sense, cosmetic materials which come into contact with skin on use, for example, skin milk, skin cream, foundation cream, massage cream, cleansing cream, shaving cream, cleansing foam, skin lotion, lotion, pack, shampoo, rinse, hair restorer, hair nourishment, hair dye, hair conditioner, toothpaste, gargle, permanent waving agent, ointment, bath preparation and body soap. The user may be any user irrespective of sex or age.

In the skin preparation for external application of the present invention, ingredients commonly used in skin preparations for external application can be blended within the range of not impairing the effect of the present invention. Examples thereof include chemicals described in Japanese Standards of Cosmetic Ingredients (JSCI), 2nd Edition, Annotation, compiled by Nippon Koteisho Kyokai, issued by Yakuji Nippo, Ltd. (1984), Specifications of Ingredient Other Than Those Listed in JSCI, supervised by Examination Division, Pharmaceutical Affairs Bureau, Ministry of Health and Welfare, issued by Yakuji Nippo, Ltd. (1993), Specifications of Ingredient Other Than Those Listed in JSCI, Supplement, supervised by Examination Division, Pharmaceutical Affairs Bureau, Ministry of Health and Welfare, issued by Yakuji Nippo, Ltd. (1993), The Comprehensive Licensing Standards of Cosmetics by Category, supervised by Examination Division, Pharmaceutical Affairs Bureau, Ministry of Health and Welfare, issued by Yakuji Nippo, Ltd. (1993), and Kesho-hin Genryo Jiten (Handbook of Cosmetic Ingredients), Nikko Chemicals (1991).

### EXAMPLES

The present invention is described in greater detail below by referring to Examples, however, the present invention is not limited to these Examples. In Examples, the amount blended is in the unit of mass %.

### Example 1

Lotion 1

| | |
|---|---|
| 1) α-Tocopherol glycine ester hydrochloride | 2.00 |
| 2) α-Tocopherol acetate | — |
| 3) Ethanol | 5.00 |
| 4) Propylene glycol | 5.00 |
| 5) Methyl parahydroxybenzoate | 0.20 |
| 6) Purified water | 87.8 |

Ingredients 1) and 3) to 5) were uniformly dispersed and dissolved and the resulting solution was added to 6) with stirring to obtain the objective lotion 1.

### Comparative Example 1

Lotion 2

| 1) α-Tocopherol glycine ester hydrochloride | — |
|---|---|
| 2) α-Tocopherol acetate | 2.00 |
| 3) Ethanol | 5.00 |
| 4) Propylene glycol | 5.00 |
| 5) Methyl parahydroxybenzoate | 0.20 |
| 6) Purified water | 87.8 |

(Production Method of Comparative Example 1)

Ingredients 2) and 3) to 5) were uniformly dispersed and dissolved and the resulting solution was added to 6) with stirring to obtain the objective lotion 2.

(Results)

Lotion 1 obtained in Example 1 was uniformly dissolved and exhibited good aging stability. On the other hand, in Comparative Example 1, uniform dissolution or dispersion could not be attained and a lotion having excellent solubility could not be obtained.

### Example 2

Lotion 3

| | |
|---|---|
| 1) α-Tocopherol glycine ester hydrochloride | 0.10 |
| 2) α-Tocopherol acetate | — |
| 3) Propylene glycol | 5.00 |
| 4) Methyl parahydroxybenzoate | 0.20 |
| 5) Purified water | 94.7 |

(Production Method of Example 2)

Ingredients 1), 3) and 4) were uniformly dispersed and dissolved and the resulting solution was added to 5) with stirring to obtain the objective lotion 3.

### Comparative Example 2

Lotion 4

| 1) α-Tocopherol glycine ester hydrochloride | — |
|---|---|
| 2) α-Tocopherol acetate | 0.10 |
| 3) Propylene glycol | 5.00 |
| 4) Methyl parahydroxybenzoate | 0.20 |
| 5) Purified water | 94.7 |

(Production Method of Comparative Example 2)

Ingredients 2), 3) and 4) were uniformly dispersed and dissolved and the resulting solution was added to 5) with stirring to obtain the objective lotion 4.

(Results)

Lotion 3 obtained in Example 2 was uniformly dissolved and exhibited good aging stability. On the other hand, in Comparative Example 2, uniform dissolution or dispersion could not be attained, floating of oil droplets was confirmed and a lotion having excellent solubility could not be obtained.

### Example 3

Lotion 5

| | |
|---|---|
| 1) α-Tocopherol glycine ester hydrochloride | 0.10 |
| 2) α-Tocopherol acetate | — |
| 3) Propylene glycol | 5.00 |
| 4) Methyl parahydroxybenzoate | 0.20 |
| 5) Magnesium or sodium ascorbyl phosphate | 3.00 |
| 6) Purified water | 92.7 |

(Production Method of Example 3)

Ingredients 1), 3) and 4) were uniformly dispersed and dissolved and the resulting solution was added with stirring to 6) in which 5) was previously dissolved, to obtain the objective lotion 5.

### Comparative Example 3

Lotion 6

| 1) α-Tocopherol glycine ester hydrochloride | — |
|---|---|
| 2) α-Tocopherol acetate | 0.10 |
| 3) Propylene glycol | 5.00 |
| 4) Methyl parahydroxybenzoate | 0.20 |
| 5) Magnesium or sodium ascorbyl phosphate | 3.00 |
| 6) Purified water | 92.7 |

(Production Method of Comparative Example 3)

Ingredients 2), 3) and 4) were uniformly dispersed and dissolved and the resulting solution was added with stirring to 6) in which 5) was previously dissolved, to obtain the objective lotion 6.

(Results)

Lotion 5 obtained in Example 3 was uniformly dissolved and exhibited good aging stability. On the other hand, in Comparative Example 3, uniform dissolution or dispersion could not be attained, floating of oil droplets was confirmed and a lotion having excellent solubility could not be obtained.

### Example 4

Gel Preparation 1 for External Application

| | |
|---|---|
| 1) α-Tocopherol glycine ester hydrochloride | 10.0 |
| 2) α-Tocopherol acetate | — |
| 3) Glycerin | 20.0 |
| 4) Octyldodecyl myristate | 70.0 |

(Production Method of Example 4)

Ingredient 1) was uniformly dispersed in 3) and the resulting dispersion was added to 4) with stirring to obtain the objective gel preparation 1 for external application.

### Comparative Example 4

Gel Preparation 2 for External Application

| 1) α-Tocopherol glycine ester hydrochloride | — |
|---|---|
| 2) α-Tocopherol acetate | 10.0 |
| 3) Glycerin | 20.0 |
| 4) Octyldodecyl myristate | 70.0 |

(Production Method of Comparative Example 4)

Ingredient 2) was uniformly dispersed in 3) and the resulting dispersion was added to 4) with stirring to obtain the objective gel preparation 2 for external application.

(Results)

Gel preparation 1 for external application obtained in Example 4 had a translucent gel appearance and exhibited good aging stability. On the other hand, in Comparative Example 4, gel was not formed.

### Example 5

Milky Lotion 1

| | |
|---|---|
| 1) α-Tocopherol glycine ester hydrochloride | 5.00 |
| 2) α-Tocopherol acetate | — |
| 3) Propylene glycol | 10.0 |
| 4) Methyl parahydroxybenzoate | 0.20 |
| 5) Methylphenylpolysiloxane | 20.0 |
| 6) Purified water | 64.8 |

(Production Method of Example 5)

Ingredient 1) was uniformly dispersed and dissolved in 3) to 5) and the resulting solution is added to 6) with stirring to obtain the objective milky lotion 1.

### Comparative Example 5

Milky Lotion 2

| 1) α-Tocopherol glycine ester hydrochloride | — |
|---|---|
| 2) α-Tocopherol acetate | 5.00 |
| 3) Propylene glycol | 10.0 |
| 4) Methyl parahydroxybenzoate | 0.20 |
| 5) Methylphenylpolysiloxane | 20.0 |
| 6) Purified water | 64.8 |

(Production Method of Comparative Example 5)

Ingredient 2) was uniformly dispersed and dissolved in 3) to 5) and the resulting solution is added to 6) with stirring to obtain the objective milky lotion 2.

(Results)

Milky lotion 1 obtained in Example 5 gave good feeling on use and exhibited good aging stability. On the other hand, in Comparative Example 5, emulsion was not formed and a milky lotion could not be obtained.

### Example 6

Milky Lotion 3

| | |
|---|---|
| 1) α-Tocopherol glycine ester hydrochloride | 5.00 |
| 2) α-Tocopherol acetate | — |
| 3) Propylene glycol | 10.0 |
| 4) Methyl parahydroxybenzoate | 0.20 |
| 5) Methylphenylpolysiloxane | 20.0 |
| 6) Magnesium or sodium ascorbyl phosphate | 3.00 |
| 7) Purified water | 62.8 |

(Production Method of Example 6)

Ingredient 1) was uniformly dispersed and dissolved in 3) to 5) and the resulting solution was added with stirring to 7) in which 6) was previously dissolved, to obtain the objective milky lotion 3.

### Comparative Example 6

Milky Lotion 4

| 1) α-Tocopherol glycine ester hydrochloride | — |
|---|---|
| 2) α-Tocopherol acetate | 5.00 |
| 3) Propylene glycol | 10.0 |
| 4) Methyl parahydroxybenzoate | 0.20 |
| 5) Methylphenylpolysiloxane | 20.0 |
| 6) Magnesium or sodium ascorbyl phosphate | 3.00 |
| 7) Purified water | 62.8 |

(Production Method of Comparative Example 6)

Ingredient 2) was uniformly dispersed and dissolved in 3) to 5) and the resulting solution was added with stirring to 7) in which 6) was previously dissolved, to obtain the objective milky lotion 4.

(Results)

Milky lotion 3 obtained in Example 6 gave good feeling on use and exhibited good aging stability. On the other hand, in Comparative Example 6, emulsion was not formed and a milky lotion could not be obtained.

### Example 7

Milky Lotion 5

| | |
|---|---|
| 1) α-Tocopherol glycine ester hydrochloride | 5.00 |
| 2) α-Tocopherol acetate | — |
| 3) Hydrogenated soybean phospholipid | 10.0 |
| 4) Methyl parahydroxybenzoate | 0.20 |
| 5) 2-Ethylhexanoic acid triglyceride | 20.0 |
| 6) Magnesium or sodium ascorbyl phosphate | 2.00 |
| 7) Purified water | 62.8 |

(Production Method of Example 7)

Ingredient 1) was uniformly dispersed and dissolved in 3) to 5) and thereto, 7) in which 6) was previously dissolved was added with stirring to obtain the objective milky lotion 5.

### Comparative Example 7

Milky Lotion 6

| 1) α-Tocopherol glycine ester hydrochloride | — |
|---|---|
| 2) α-Tocopherol acetate | 5.00 |
| 3) Hydrogenated soybean phospholipid | 10.0 |
| 4) Methyl parahydroxybenzoate | 0.20 |
| 5) 2-Ethylhexanoic acid triglyceride | 20.0 |
| 6) Magnesium or sodium ascorbyl phosphate | 2.00 |
| 7) Purified water | 62.8 |

(Production Method of Comparative Example 7)

Ingredient 2) was uniformly dispersed and dissolved in 3) to 5) and the resulting solution is added, with stirring, to 7) in which 6) was previously dissolved to obtain the objective milky lotion 6.

(Results)

Milky lotion 5 obtained in Example 7 gave good feeling on use and exhibited good aging stability. On the other hand, in Comparative Example 7, although a milky lotion was obtained, phase separation was observed after a few days and good aging stability could not be obtained.

### Example 8 and Comparative Example 8

Evaluation of Skin Penetrability

(Method)

In each of φ35 mm plastic Petri dishes, 1 ml of a Dulbecco's MEM medium containing 1), 2) or 3) was placed and a nylon mesh and a lens paper were sequentially laid thereon. On the lens paper, a skin removed from the back of a hairless mouse was placed such that the epidermis came into contact with the lens paper. At this time, the dermis side was covered with a parafilm and thereby prevented from drying.

| 1) Not added | |
|---|---|
| 2) α-Tocopherol glycine ester hydrochloride | 0.50 |
| 3) α-Tocopherol acetate | 0.50 |

After the passage of 4 hours at 37° C., the skin was washed with a phosphoric acid buffer solution and homogenized. Then, the amount of α-tocopherol in the skin was measured. The determination of α-tocopherol was performed by high performance liquid chromatography.

The conditions for measurement by high performance liquid chromatography were as follows.
Column: Shodex ODSpak F-411
Temperature: 40° C.
Eluent: methanol/acetonitrile=7/3 (containing 0.02M acetic acid and 0.02M sodium acetate)
Flow rate: 0.7 ml
Detection: fluorescent, Ex: 298 nm, Em: 325 nm

(Results)
1) 10 nmol/g of skin
2) 24 nmol/g of skin
3) 11 nmol/g of skin

The skin treated with α-tocopherol glycine ester hydrochloride had significant increase of the α-tocopherol amount.

### Example 9 and Comparative Example 9

Conversion to α-Tocopherol in Keratinocyte of Human

Epidermis

(Method)

Commercially available keratinocytes of normal human epidermis were cultured in the medium attached. The cells were harvested and spalled by freeze-thawing method. To this cell spall solution, 1), 2) or 3) was added to have a final concentration of 1 mM. The resulting solution was kept at 37° C. for 2 hours and then the amount of α-tocopherol liberated in the reaction solution was measured. The determination of α-tocopherol was performed by high performance liquid chromatography under the same conditions as in Example 8 and Comparative Example 8.
1) Not added
2) α-Tocopherol glycine ester hydrochloride
3) α-Tocopherol acetate

(Results)
1) lower than detection limit
2) 37.5 nmol/ml of cell suspension
3) 0.5 nmol/ml of cell suspension

The cell spall solution in which α-tocopherol glycine ester hydrochloride was added, had significant increase of the α-tocopherol amount.

### Example 10

Lotion 7

| | |
|---|---|
| 1) γ-Tocopherol glycine ester hydrochloride | 2.00 |
| 2) γ-Tocopherol acetate | — |
| 3) Ethanol | 5.00 |
| 4) Propylene glycol | 5.00 |
| 5) Methyl parahydroxybenzoate | 0.20 |
| 6) Purified water | 87.8 |

(Production Method of Example 10)

Ingredients 1) and 3) to 5) were uniformly dispersed and dissolved and the resulting solution was added to 6) with stirring to obtain the objective lotion 7.

### Comparative Example 10

Lotion 8

| 1) γ-Tocopherol glycine ester hydrochloride | — |
|---|---|
| 2) γ-Tocopherol acetate | 2.00 |
| 3) Ethanol | 5.00 |
| 4) Propylene glycol | 5.00 |
| 5) Methyl parahydroxybenzoate | 0.20 |
| 6) Purified water | 87.8 |

(Production Method of Comparative Example 10)

Ingredients 2) and 3) to 5) were uniformly dispersed and dissolved and the resulting solution was added to 6) with stirring to obtain the objective lotion 8.

(Results)

Lotion 7 obtained in Example 10 was uniformly dissolved and exhibited good aging stability. On the other hand, in Comparative Example 10, uniform dissolution or dispersion could not be attained and a lotion having excellent solubility could not be obtained.

### Example 11

Lotion 9

| | |
|---|---|
| 1) γ-Tocopherol glycine ester hydrochloride | 0.10 |
| 2) γ-Tocopherol acetate | — |
| 3) Propylene glycol | 5.00 |
| 4) Methyl parahydroxybenzoate | 0.20 |
| 5) Purified water | 94.7 |

(Production Method of Example 11)

Ingredients 1), 3) and 4) were uniformly dispersed and dissolved and the resulting solution was added to 5) with stirring to obtain the objective lotion 9.

### Comparative Example 11

Lotion 10

| 1) γ-Tocopherol glycine ester hydrochloride | — |
|---|---|
| 2) γ-Tocopherol acetate | 0.10 |
| 3) Propylene glycol | 5.00 |
| 4) Methyl parahydroxybenzoate | 0.20 |
| 5) Purified water | 94.7 |

(Production Method of Comparative Example 11)

Ingredients 2), 3) and 4) were uniformly dispersed and dissolved and the resulting solution was added to 5) with stirring to obtain the objective lotion 10.

(Results)

Lotion 9 obtained in Example 11 was uniformly dissolved and exhibited good aging stability. On the other hand, in Comparative Example 11, uniform dissolution or dispersion could not be attained, floating of oil droplets was confirmed and a lotion having excellent solubility could not be obtained.

### Example 12

Lotion 11

| | |
|---|---|
| 1) γ-Tocopherol glycine ester hydrochloride | 0.10 |
| 2) γ-Tocopherol acetate | — |
| 3) Propylene glycol | 5.00 |
| 4) Methyl parahydroxybenzoate | 0.20 |
| 5) Magnesium or sodium ascorbyl phosphate | 3.00 |
| 6) Purified water | 92.7 |

(Production Method of Example 12)

Ingredients 1), 3) and 4) were uniformly dispersed and dissolved and the resulting solution was added with stirring to 6) in which 5) was previously dissolved, to obtain the objective lotion 11.

### Comparative Example 12

Lotion 12

| 1) γ-Tocopherol glycine ester hydrochloride | — |
|---|---|
| 2) γ-Tocopherol acetate | 0.10 |
| 3) Propylene glycol | 5.00 |
| 4) Methyl parahydroxybenzoate | 0.20 |
| 5) Magnesium or sodium ascorbyl phosphate | 3.00 |
| 6) Purified water | 92.7 |

(Production Method of Comparative Example 12)

Ingredients 2), 3) and 4) were uniformly dispersed and dissolved and the resulting solution was added with stirring to 6) in which 5) was previously dissolved, to obtain the objective lotion 12.

(Results)

Lotion 11 obtained in Example 12 was uniformly dissolved and exhibited good aging stability. On the other hand, in Comparative Example 12, uniform dissolution or dispersion could not be attained, floating of oil droplets was confirmed and a lotion having excellent solubility could not be obtained.

### Example 13

Gel Preparation 3 for External Application

| | |
|---|---|
| 1) γ-Tocopherol glycine ester hydrochloride | 10.0 |
| 2) γ-Tocopherol acetate | — |
| 3) Glycerin | 20.0 |
| 4) Octyldodecyl myristate | 70.0 |

(Production Method of Example 13)

Ingredient 1) was uniformly dispersed in 3) and the resulting dispersion was added to 4) with stirring to obtain the objective gel preparation 3 for external application.

### Comparative Example 13

Gel Preparation 4 for External Application

| 1) γ-Tocopherol glycine ester hydrochloride | — |
|---|---|
| 2) γ-Tocopherol acetate | 10.0 |
| 3) Glycerin | 20.0 |
| 4) Octyldodecyl myristate | 70.0 |

(Production Method of Comparative Example 13)

Ingredient 2) was uniformly dispersed in 3) and the resulting dispersion was added to 4) with stirring to obtain the objective gel preparation 4 for external application.

(Results)

Gel preparation 3 for external application obtained in Example 13 had a translucent gel appearance and exhibited good aging stability. On the other hand, in Comparative Example 13, gel was not formed.

### Example 14

Milky Lotion 7

| | |
|---|---|
| 1) γ-Tocopherol glycine ester hydrochloride | 5.00 |
| 2) γ-Tocopherol acetate | — |
| 3) Propylene glycol | 10.0 |
| 4) Methyl parahydroxybenzoate | 0.20 |
| 5) Methylphenylpolysiloxane | 20.0 |
| 6) Purified water | 64.8 |

(Production Method of Example 14)

Ingredient 1) was uniformly dispersed and dissolved in 3) to 5) and the resulting solution was added to 6) with stirring to obtain the objective milky lotion 7.

### Comparative Example 14

Milky Lotion 8

| 1) γ-Tocopherol glycine ester hydrochloride | — |
|---|---|
| 2) γ-Tocopherol acetate | 5.00 |
| 3) Propylene glycol | 10.0 |
| 4) Methyl parahydroxybenzoate | 0.20 |
| 5) Methylphenylpolysiloxane | 20.0 |
| 6) Purified water | 64.8 |

(Production Method of Comparative Example 14)

Ingredient 2) was uniformly dispersed and dissolved in 3) to 5) and the resulting solution was added to 6) with stirring to obtain the objective milky lotion 8.

(Results)

Milky lotion 7 obtained in Example 14 gave good feeling on use and exhibited good aging stability. On the other hand, in Comparative Example 14, emulsion was not formed and a milky lotion could not be obtained.

### Example 15

Milky Lotion 9

| | |
|---|---|
| 1) γ-Tocopherol glycine ester hydrochloride | 5.00 |
| 2) γ-Tocopherol acetate | — |
| 3) Propylene glycol | 10.0 |
| 4) Methyl parahydroxybenzoate | 0.20 |
| 5) Methylphenylpolysiloxane | 20.0 |
| 6) Magnesium or sodium ascorbyl phosphate | 3.00 |
| 7) Purified water | 62.8 |

(Production Method of Example 15)

Ingredient 1) was uniformly dispersed and dissolved in 3) to 5) and the resulting solution was added with stirring to 7) in which 6) was previously dissolved, to obtain the objective milky lotion 9.

### Comparative Example 15

Milky Lotion 10

| 1) γ-Tocopherol glycine ester hydrochloride | — |
|---|---|
| 2) γ-Tocopherol acetate | 5.00 |
| 3) Propylene glycol | 10.0 |
| 4) Methyl parahydroxybenzoate | 0.20 |
| 5) Methylphenylpolysiloxane | 20.0 |
| 6) Magnesium or sodium ascorbyl phosphate | 3.00 |
| 7) Purified water | 62.8 |

(Production Method of Comparative Example 15)

Ingredient 2) was uniformly dispersed and dissolved in 3) to 5) and the resulting solution was added with stirring to 7) in which 6) was previously dissolved, to obtain the objective milky lotion 10.

(Results)

Milky lotion 9 obtained in Example 15 gave good feeling on use and exhibited good aging stability. On the other hand, in Comparative Example 15, emulsion was not formed and a milky lotion could not be obtained.

### Example 16

Milky Lotion 11

| | |
|---|---|
| 1) γ-Tocopherol glycine ester hydrochloride | 5.00 |
| 2) γ-Tocopherol acetate | — |
| 3) Hydrogenated soybean phospholipid | 10.0 |
| 4) Methyl parahydroxybenzoate | 0.20 |
| 5) 2-Ethylhexanoic acid triglyceride | 20.0 |
| 6) Magnesium or sodium ascorbyl phosphate | 2.00 |
| 7) Purified water | 62.8 |

(Production Method of Example 16)

Ingredient 1) was uniformly dispersed and dissolved in 3) to 5) and the resulting solution was added, with stirring, to 7) in which 6) was previously dissolved to obtain the objective milky lotion 11.

### Comparative Example 16

Milky Lotion 12

| 1) γ-Tocopherol glycine ester hydrochloride | — |
|---|---|
| 2) γ-Tocopherol acetate | 5.00 |
| 3) Hydrogenated soybean phospholipid | 10.0 |
| 4) Methyl parahydroxybenzoate | 0.20 |
| 5) 2-Ethylhexanoic acid triglyceride | 20.0 |
| 6) Magnesium or sodium ascorbyl phosphate | 2.00 |
| 7) Purified water | 62.8 |

(Production Method of Comparative Example 16)

Ingredient 2) was uniformly dispersed and dissolved in 3) to 5) and the resulting solution was added, with stirring, to 7) in which 6) was previously dissolved to obtain the objective milky lotion 12.

(Results)

Milky lotion 11 obtained in Example 16 gave good feeling on use and exhibited good aging stability. On the other hand, in Comparative Example 16, although a milky lotion was obtained, phase separation was observed after a few days and good aging stability could not be obtained. Example 17 and Comparative Example 17 Evaluation of Skin Penetrability

(Method)

In each of φ35 mm plastic Petri dishes, 1 ml of a Dulbecco's MEM medium containing 1), 2) or 3) was placed and a nylon mesh and a lens paper were sequentially laid thereon. On the lens paper, a skin removed from the back of a hairless mouse was placed such that the epidermis came into contact with the lens paper. At this time, the dermis side was covered with a parafilm and thereby prevented from drying.

| 1) Not added | |
|---|---|
| 2) γ-Tocopherol glycine ester hydrochloride | 0.50 |
| 3) γ-Tocopherol acetate | 0.50 |

After the passage of 4 hours at 37° C., the skin was washed with a phosphoric acid buffer solution and homogenized. Then, the amount of γ-tocopherol in the skin was measured. The determination of γ-tocopherol was performed by high performance liquid chromatography.

The conditions for measurement by high performance liquid chromatography were as follows.
Column: Shodex ODSpak F-411
Temperature: 40° C.
Eluent: methanol/acetonitrile=7/3 (containing 0.02M acetic acid and 0.02M sodium acetate)
Flow rate: 0.7 ml
Detection: fluorescent, Ex: 298 nm, Em: 325 nm
   (Results)
1) 10 nmol/g of skin
2) 24 nmol/g of skin
3) 11 nmol/g of skin

The skin treated with γ-tocopherol glycine ester hydrochloride had significant increase of the γ-tocopherol amount.

### Example 18 and Comparative Example 18

Conversion to γ-Tocopherol in Keratinocyte of Human Epidermis

(Method)

Commercially available keratinocytes of normal human epidermis were cultured in the medium attached. The cells were harvested and spalled by freeze-thawing method. To this cell spall solution, 1), 2) or 3) was added to have a final concentration of 1 mM. The resulting solution was kept at 37° C. for 2 hours and then the amount of γ-tocopherol liberated in the reaction solution was measured. The determination of γ-tocopherol was performed by high performance liquid chromatography under the same conditions as in Example 17 and Comparative Example 17.
1) Not added
2) γ-Tocopherol glycine ester hydrochloride
3) γ-Tocopherol acetate

(Results)
1) lower than detection limit
2) 37.5 nmol/ml of cell suspension
3) 0.5 nmol/ml of cell suspension

The cell spall solution in which γ-tocopherol glycine ester hydrochloride was added, had significant increase of the γ-tocopherol amount.

### Example 19

Lotion 13

| | |
|---|---|
| 1) α-tocopherol glycine ester hydrocloride | 2.00 |
| 2) α-tocopherol glycine ester | — |
| 3) ethanol | 5.00 |
| 4) propylene glycol | 5.00 |
| 5) methyl p-hydroxybenzoate | 0.20 |
| 6) purified water | 87.8 |

(Production Method of Example 19)

1) and 3) to 5) were uniformly dispersed in a solution and this was added to 6) with stirring to obtain the target lotion 13.

### Comparative Example 19

Lotion 14

| 1) α-tocopherol glycine ester hydrocloride | — |
|---|---|
| 2) α-tocopherol glycine ester | 2.00 |
| 3) ethanol | 5.00 |
| 4) propylene glycol | 5.00 |
| 5) methyl p-hydroxybenzoate | 0.20 |
| 6) purified water | 87.8 |

(Production Method of Comparative Example 19)

2) and 3) to 5) were uniformly dispersed in a solution and this was added to 6) with stirring to obtain the target lotion 14.

(Results)

The lotion 13 obtained in Example 19 was uniformly dissolved; and had good stability over time. In contrast, the lotion of Comparative Example 19 could not be uniformly dissolved or dispersed, and the presence of a suspension of oil drops was confirmed, and a lotion having excellent solubility could not be obtained.

### Example 20

Gel external use agent 5

| | |
|---|---|
| 1) α-tocopherol glycine ester hydrocloride | 10.0 |
| 2) α-tocopherol glycine ester | — |
| 3) glycerin | 20.0 |
| 4) octyldodecyl myristate | 70.0 |

(Production Method of Example 20)

1) was uniformly dispersed in 3), and this was added to 4) with stirring to obtain the target gel external use agent 5.

### Comparative Example 20

Gel external use agent 6

| 1) α-tocopherol glycine ester hydrocloride | — |
|---|---|
| 2) α-tocopherol glycine ester | 10.0 |
| 3) glycerin | 20.0 |
| 4) octyldodecyl myristate | 70.0 |

(Production Method of Comparative Example 20)

2) was uniformly dispersed in 3), and this was added to 4) with stirring to obtain the target gel external use agent 6.

(Results)

The gel external use agent obtained in Example 20 was a gel having a translucent appearance, and showed good stability over time. In contrast, a gel could not be formed in Comparative Example 20.

### Example 21

Milky Lotion 13

| | |
|---|---|
| 1) α-tocopherol glycine ester hydrocloride | 5.00 |
| 2) α-tocopherol acetate | — |
| 3) propylene glycol | 10.0 |
| 4) methyl p-hydroxybenzoate | 0.20 |
| 5) methylphenyl polysiloxane | 20.0 |
| 6) purified water | 64.8 |

(Production Method of Example 21)

1) was uniformly dispersed and dissolved in 3) to 5), and the resultant solution was added to 6) with stirring to obtain the target emulsion 13.

### Comparative Example 21

Milky Lotion 14

| 1) α-tocopherol glycine ester hydrocloride | — |
|---|---|
| 2) α-tocopherol acetate | 5.00 |
| 3) propylene glycol | 10.0 |
| 4) methyl p-hydroxybenzoate | 0.20 |
| 5) methylphenyl polysiloxane | 20.0 |
| 6) purified water | 64.8 |

(Production Method of Comparative Example 21)

2) was uniformly dispersed and dissolved in 3) to 5), and this was added to 6) with stirring to obtain the target emulsion 13.

(Results)

The emulsion 13 obtained in Example 21 had a good feel in use, and showed good stability over time. In contrast, an emulsion could not be formed in Comparative Example 21, and a milky lotion could not be obtained.

### Example 22

Lotion 15

| | |
|---|---|
| 1) γ-tocopherol glycine ester hydrocloride | 2.00 |
| 2) γ-tocopherol glycine ester | — |
| 3) ethanol | 5.00 |
| 4) propylene glycol | 5.00 |
| 5) methyl p-hydroxybenzoate | 0.20 |
| 6) purified water | 87.8 |

(Production Method of Example 22)

1) and 3) to 5) were uniformly dispersed in a solution and this was added to 6) with stirring to obtain the target lotion 15.

### Comparative Example 22

Lotion 16

| 1) γ-tocopherol glycine ester hydrocloride | — |
|---|---|
| 2) γ-tocopherol glycine ester | 2.00 |
| 3) ethanol | 5.00 |
| 4) propylene glycol | 5.00 |
| 5) methyl p-hydroxybenzoate | 0.20 |
| 6) purified water | 87.8 |

(Production Method of Comparative Example 22)

2) and 3) to 5) were uniformly dispersed in a solution and this was added to 6) with stirring to obtain the target lotion 16.

(Results)

The lotion 15 obtained in Example 22 was uniformly dissolved, and had good stability over time. In contrast, the lotion of Comparative Example 22 could not be uniformly dissolved or dispersed, and a lotion having excellent solubility could not be obtained.

### Example 23

Gel external use agent 7

| | |
|---|---|
| 1) γ-tocopherol glycine ester hydrocloride | 10.0 |
| 2) γ-tocopherol glycine ester | — |
| 3) glycerin | 20.0 |
| 4) octyldodecyl myristate | 70.0 |

(Production Method of Example 23)

1) was uniformly dispersed in 3), and this was added to 4) with stirring to obtain the target gel external use agent 7.

### Comparative Example 23

Gel external use agent 8

| 1) γ-tocopherol glycine ester hydrocloride | — |
|---|---|
| 2) γ-tocopherol glycine ester | 10.0 |
| 3) glycerin | 20.0 |
| 4) octyldodecyl myristate | 70.0 |

(Production Method of Comparative Example 23)

2) was uniformly dispersed in 3), and this was added to 4) with stirring to obtain the target gel external use agent 8.

(Results)

The gel external use agent obtained in Example 23 was a gel having a translucent appearance, and showed good stability over time. In contrast, a gel could not be formed in Comparative Example 23.

### Example 24

Milky Lotion 15

| | |
|---|---|
| 1) γ-tocopherol glycine ester hydrocloride | 5.00 |
| 2) γ-tocopherol acetate | — |
| 3) propylene glycol | 10.0 |
| 4) methyl p-hydroxybenzoate | 0.20 |
| 5) methylphenyl polysiloxane | 20.0 |
| 6) purified water | 64.8 |

(Production Method of Example 24)

1) was uniformly dispersed and dissolved in 3) to 5), and this was added to 6) with stirring to obtain the target emulsion 15.

### Comparative Example 24

Emulsion 16

| 1) γ-tocopherol glycine ester hydrocloride | — |
|---|---|
| 2) γ-tocopherol acetate | 5.00 |
| 3) propylene glycol | 10.0 |
| 4) methyl p-hydroxybenzoate | 0.20 |
| 5) methylphenyl polysiloxane | 20.0 |
| 6) purified water | 64.8 |

(Production Method of Comparative Example 24)

2) was uniformly dispersed and dissolved in 3) to 5), and this was added to 6) with stirring to obtain the target milky lotion 16.

(Results)

The milky lotion 15 obtained in Example 24 had a good feel in use, and showed good stability over time. In contrast, an emulsion could not be formed in Comparative Example 24, and an emulsion could not be obtained.

### INDUSTRIAL APPLICABILITY OF THE INVENTION

The tocopherol aminoalkylcarboxylate ester having no substituent on the N atom and/or a salt thereof of the present invention are excellent in solubility and emulsifiability as compared with tocopherols in a simple form. Furthermore, when applied as a skin preparation for external application, these are readily hydrolyzed by an esterase or carboxyl esterase in the skin tissue to produce an active free tocopherol and therefore, can be used as an active ingredient of skin preparations for external application which are expected to have efficacy and effect such as activities of antioxidation, vital membrane stabilization, immunoactivation and acceleration of blood circulation, and also can be suitably used as a cosmetic material or the like.

1. A skin preparation for external application, comprising a tocopherol aminoalkylcarboxylate ester having no substituent on the N atom and/or a salt thereof.

2. The skin preparation for external application according to claim 1, wherein the tocopherol aminoalkylcarboxylate ester is one or more compound selected from α-tocopherol derivatives, β-tocopherol derivatives, γ-tocopherol derivatives and δ-tocopherol derivatives.

3. The skin preparation for external application according to claim 2, wherein the tocopherol aminoalkylcarboxylate ester is an α-tocopherol aminoalkylcarboxylate ester or a γ-tocopherol aminoalkylcarboxylate ester.

4. The skin preparation for external application according to claim 1, wherein the tocopherol aminoalkylcarboxylate ester having no substituent on the N atom comprises a compound represented by the following formula (I): 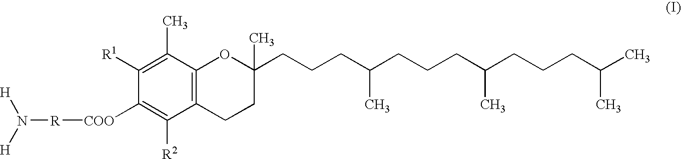
(wherein R¹ and R² each represents a hydrogen atom or a methyl group and R represents a branched or linear alkylene group which may have a substituent).

5. The skin preparation for external application according to claim 1, wherein the tocopherol aminoalkylcarboxylate ester having no substituent on the N atom comprises a compound represented by the following formula (II): 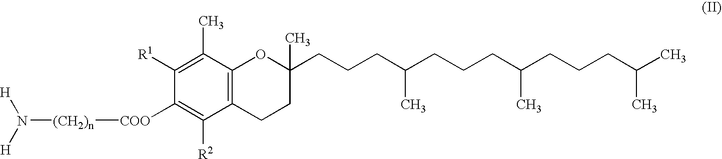
(wherein R¹ and R² each represents a hydrogen atom or a methyl group, and n represents an integer of 1 to 7).

6. The skin preparation for external application according to claim 1, wherein the salt is a hydrohalogenic acid.

7. The skin preparation for external application according to claim 1, wherein the salt is an organic acid salt.

8. The skin preparation for external application according to claim 6, wherein the hydrohalogenic acid is hydrochloric acid.

9. The skin preparation for external application according to claim 1, wherein the content of the tocopherol aminoalkylcarboxylate ester having no substituent on the N atom and/or a salt thereof is from 0.01 to 10 mass %.

10 The skin preparation for external application according to claim 1, wherein the tocopherol aminoalkylcarboxylate ester and/or a salt thereof is tocopherol glycine ester and/or a salt thereof.

11. A cosmetic material comprising the skin preparation for external application according to claim 1.
